(19) United States
(12) Patent Application Publication
(10) Pub. No.: US 2024/0355432 A1
(43) Pub. Date: Oct. 24, 2024

(54) SYSTEM AND METHOD OF MEDICAL DATA ENTRY

(71) Applicant: Rachel Gologorsky, Miami Beach, FL (US)

(72) Inventor: Rachel Gologorsky, Miami Beach, FL (US)

(21) Appl. No.: 18/305,362

(22) Filed: Apr. 23, 2023

(51) Int. Cl. G16H 10/20; G06F 40/174; G06V 30/416; G16H 10/60; G16H 40/67

(52) U.S. Cl. CPC G16H 10/20; G06F 40/174; G06V 30/416; G16H 10/60; G16H 40/67; G16H 50/20

(57) ABSTRACT

A processor executed method for assisting a user in filling out a medical form that provides an easy way of obtaining the medical form contents from its native source, processing the contents to identify missing information and then providing suggestions for filling in the missing information in the form from previously stored information of the patient or by acquiring the missing information through the system sensors and questions posed to the patient. The present invention provides a practical method of improving and automating user interaction with a variety of medical systems and paper forms that also enhances the accuracy and availability of the relevant information when it is needed by a health care provider.

### BACKGROUND

An accurate and complete electronic medical record (EMR) is key to maintaining a patient's health and to diagnosing new medical conditions.

Monitoring technologies in hospitals and their operating theatres that have been seamlessly integrated into medical and surgical work. Digital health technologies are becoming increasingly portable and are leaving hospitals and healthcare practitioners into the hands of lay users for self-monitoring and self-care. Both human sensing and technological sensors contribute to sensemaking about health and illness, as the practitioner creates meaning in their sensed experience.

Despite advances in diagnostic procedures, eliciting the patient's history is still the most important part of the diagnostic effort. In previously undiagnosed conditions in internal medicine, the final diagnosis was reached after the patient history in 76% of the cases, after the physical examination in 12% of the cases, and after laboratory investigations in 11% of the cases.

Failure to elicit all of the concerns of the patient can lead to poorer patient satisfaction. the first complaint of the patient corresponded with the final diagnosis for 76% of somatic disorders but for only 6% of psychosocial ones. Detection of psychosocial problems is a particularly difficult diagnostic task and one that family physicians face many times a day. Approximately one third of the patients consulting a primary care physician have a psychosocial problem of sufficient magnitude to impair their functioning, well-being, or both.

Certain characteristics of patients make the task of detection harder. Physicians probe more when faced with women and people in difficult social or economic circumstances and are less alert with men and those in apparently easier life conditions.

Psychosocial problems cause many somatic symptoms and patients are likely to present only the somatic symptoms to the physician.

There are several problems with the way EMR are presently handled.

Interpretation services are time-consuming when used, and so are often not used, resulting in inaccurate or incomplete HPIs. Busy healthcare staff often do not have time to verify with patients that EMR information is correct (e.g. medication list in EMR is up-to-date).

The present disclosure improves capabilities to measure, analyze, and report on a large number of symptoms of previous systems such as in US 2022/0079439 which is hereby incorporated by reference.

The present disclosure makes use of exchanging information among components of a smart health system with mobile devices, such as described in US 2022/0223286 which is hereby incorporated by reference.

The present disclosure also seeks to extend the advantages of a distributed computing system such as the systems in US 2004/0049609 hereby incorporated by reference. The concept of distributed computing can be extended to modern signal processing and AI systems to tailor processing task distribution, processing speed, data access, power consumption, and privacy to the targeted applications.

### SUMMARY

To enter history of present illness (HPI) incorporated into electronic medical record (EMR) without healthcare staff intermediary. Save time. Get comprehensive HPI. Save time in office, hospital, or ER triage for thorough interview and documentation. HPI is based on a standard set of broad questions followed by a targeted subset of pertinent positives, negatives, and risk factors associated with the initial pattern.

Patient information can be augmented by a better construction of the patient history, by measuring and evaluating results of the available sensors and diagnosis equipment, and by smart comparison of patient data to information in medical databases.

The application can provide a secondary source of information to complete or confirm the information that the patient or the physician has access to, including a better patient history and present condition information from sensory.

In a case where a patient is using third party sensors or medical monitoring devices, the system can import that information if available in a textual form or a compatible text or hypertext document format. If the information from the devices is in not available in a textual form, the system can mine the information by capturing the screens or the indicators of the devices and processing the image information to obtain the sensor data in the desired textual format. This can include optical character recognition of the displayed text or optical comparison of the imaged indicators to the known states and measurements that the indicators represent.

The application can improve Patient satisfaction and compliance by improving patient experience. Patients are more compliant with prescribed treatments when they have more information but also when they have to answer fewer questions and undergo fewer tests. Patients consistently express an interest in receiving information from the physician and are more satisfied with visits in which more information is given. Thus, the application can identify and present pertinent information and answer pertinent questions to the patient, the physician, or any other person involved, and in this way enhance the quality and the effectiveness of the human interactions as well.

### BRIEF DESCRIPTION OF THE DRAWINGS

FIG. 1 illustrates use of smart devices and networks in smart health technology.

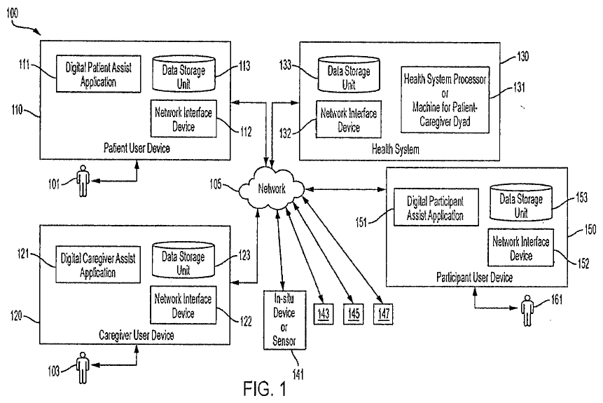

FIG. 2 illustrates an example of a processing resources distributed over a network.

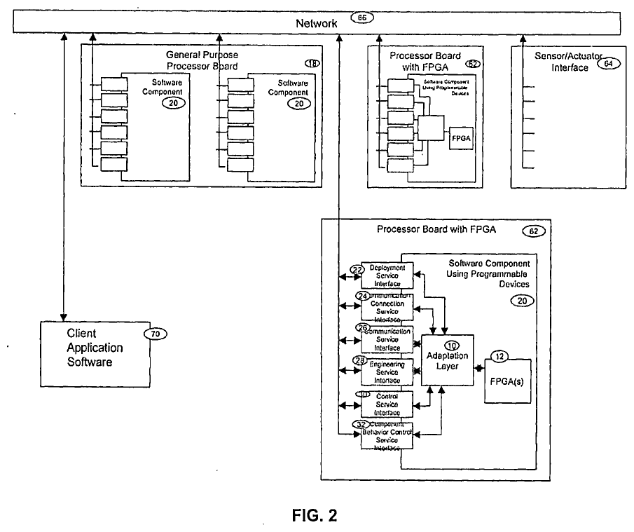

FIG. 3 illustrates integration of multiple sources of data and user access.

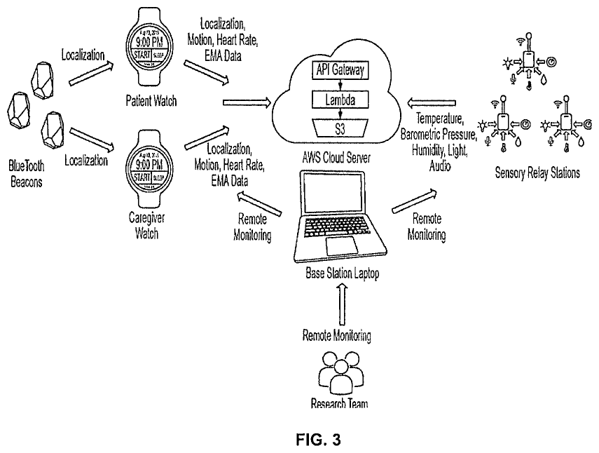

FIG. 4 illustrates integration of sensors, including an imaging system, into a computing system.

### DETAILED DESCRIPTION

To provide comprehensive HPI, guideline adherence, insurance reimbursement.

Physicians often do not have time to ask or document the full range of relevant symptoms and risk factors, a gap that this application addresses.

To provide HPI Quality & Patient convenience. Patient can answer questions in private, in their native tongue, on their own time. Non-judgmental approach to sensitive questions posed by an application—e.g. alcohol, smoking, substance use, sexual practices—may make patients more at ease. To provide factually correct HPI. To provide HPI Transparency & Real-time responsiveness. Patient has the opportunity to re-visit and update any parts in “AI HPI” in real-time.

To provide re-allocation of time with staff to most attention-worthy parts of HPI or parts requiring further elaboration.

To provide capability for AI triage of patient severity, facilitating resource-efficient patient direction: urgent care site, ER, direct-to-hospital admission. Additional AI capability may triage based on possibility of urgent diagnosis. Diagnoses such as appendicitis, heart attack, stroke have a unique set of symptoms whose presence alerts to possibility of serious condition.

The invention may extract additional information from the audio/video feed and/or various sensor readings. Recognized facial expressions and eye movements can provide information on pain severity, depression, anxiety, transient ischemic attack or stroke (presence of facial droop, facial asymmetry), Bell's palsy, etc. Eye dilation, sweating, pallor, jaundice, estimated BMI can help with diagnosis of toxin ingestion, blood loss, chronic state of health. Audio input like cough or speech pattern can help with diagnosis (monotone, flat affect, pressured speech, slurred speech, staccato or barking cough, dry or wet cough, inspiratory wheeze, shortness of breath, etc.).

The application can also elicit physical information by asking the patient to smile (evaluate facial weakness/asymmetry), protrude tongue, stand with feet together and eye closed (balance), track an object on the screen with their eyes, etc. While the patient may not be able to accurately describe or diagnose his or her own symptoms, the system can elicit accurate information from patient's automated and involuntary responses to the system's directions. The readings from various sensors, which may be individually enhanced and/or jointly enhanced, and combined in signal processing and/or AI to identify patient's symptoms.

The application can ask open ended psychological questions based on what the patient has said, make empathied comments, and interrupt less. This leads their patients to give more cues indicative of psychological disturbance.

The application can elicit additional information by giving step-by-step directions to patient's friends, family, medical professional, or another caregiver on how to identify patient's symptoms by making observations and, performing diagnostic physical maneuvers (e.g. Murphy's sign for cholecystitis), on how to check vital signs manually, on how to perform tests with available diagnosis equipment, for example, to ultrasound the patient with a point-of-care ultrasound.

For example, this methodology can be used to do a nuanced assessment of cognitive performance and screen for fall risk, dementia symptoms and progression. The application can give the patient instructions to perform cognitive tasks using the application user interface and/or physical activities such as cross-body commands, walking, gait assessment, and responding to written tests. The system can obtain and process additional data based on camera observation of the patient responses, speed of action, mood, appropriateness of appearance, appropriateness of footwear and foot hygiene, and pattern in using the input interface. The system can also assess the patient's immediate home environment for safety compliance, cleanliness, wear of the home furnishings, appropriate availability of accessibility hardware such as walking aids, grab bars, secured rugs, and so on.

This can be performed in a home setting and at a hospital to facilitate timely recognition of delirium.

Also note that while the discussion is primarily directed to human patients, a patient can refer to a human or an animal, and the diagnosis can be performed for treatment or research purposes.

The application can also provide emergency care instructions on the basis of available personnel and equipment at the moment of emergency. For example, instruct CPR providers on what to do and when, and give real-time feedback on whether they are compressing the chest at the right frequency and at the right depth.

### The Solutions can be Provided by Enhanced Technical Capabilities

A user interface. Patient and application interact via video application, where application queries are voiced and subtitled on a screen, as is patient response. Dialogue in patient preferred language; dual documentation in the country's official language and the patient preferred language. Natural language algorithm to select relevant follow-up questions for bot-patient interaction.

A module that communicates information with a new part of the patient's EMR—the “AI HPI”.

A mechanism for patient review of “AI HPI” and re-engagement with application to update “AI HPI” at any point.

Diagnosis is not the product of an isolated event of rational decision-making, but rather can be a continuous monitoring of the patient's condition and adjustment to care. In this diagnostic process there is often a back-and-forth between human sensing and a sensing of sensors. Diagnosis is not based on isolated pieces of evidence but on layers of sense that are emergent, contingent, continuing and recursive in nature.

The application AI is particularly suitable to extract diagnosis information in situations in which a full suite of sensed information is not available, such as in at-home locations or in application of telemedicine. Telemedicine covers a range of technologies that facilitate ‘virtual medical encounters’ over a network of distributed locations and actors, and features ‘remote doctors’ and ‘absent patients’. In this, diagnosis involves increased delegation of work: patients are required to ‘inspect’ their own bodies; nurses who may be physically present with the patient while the doctor is communicating remotely may also be required to engage in the kind of sensory assessments usually performed by doctors.

Improving diagnosis by information obtained by sensory, sensory processing, elicited patient information, and AI. A rule-based diagnosis procedure can often out-perform physicians in identifying clinically significant information. But existing solutions do not communicate with EMRs, or effectively interact with the user, the caretakers, or the sensory used in diagnosis.

The AI can be trained using patient data available in the present electronic records and then updated with data and sensor readings obtained while using the system. For example, the system can use audio transcripts of patient interviews in care settings, such as urgent care sites, ER, primary care, and physician office visits. Physician-testing question banks with thousands of questions with patient HPI's and matching diagnosis can be used to train AI to match symptom patterns with diagnosis.

Patient can provide information by answering direct questions, by taking specific measurements, or by reacting to eliciting questions and stimuli. For example, mental health information can be obtained by psychiatric questionnaires, depression-screening questions, word-association questions, by having patients perform directed actions, or by having patients play games. The settings of the diagnosis can be adjusted adaptively based on patient responses in order to improve diagnosis speed and accuracy. The diagnosis can stimulate changes in the state of mind and elicit emotional responses based on the determined state of mind, mental ability and other patient factors determined from patient history and sensor data.

Eye tracking data can offer quantitative assessments of fatigue, mental workload and comprehension, emotional state and emotion intensity, among other cognitive and psychiatric endpoints. Incorporating such sensor data offers the opportunity for physicians to mitigate bias with quantitative assessments of psychiatric and cognitive function, (2) to improve patient comprehension in real-time during the encounter, and (3) to evaluate and monitor emotional state during the encounter (e.g. anxiety, confusion, irritation, fear) in order to surface awareness to the physician and enhance emotional support.

Patient history can be enhanced by monitoring of, social interactions, sentiment analysis of text and voice data, exercise frequency, quantity/quality of sleep, purchase history. This can be done by authorized access to patient's social media data, financial data such as credit card purchases, and health data such as from wearable sensors.

The system can also distract patients to gauge true level of pain, vitals, etc. This is useful to mitigate biases in self-perception, such as cultural biases, biases due to embarrassment or pride, biases due to “white-coat effect” and so on.

Using biometrics to provide additional diagnosis data. For example, pain assessment can be improved by monitoring pupil dilation, blink rate, sweating, micro expressions, time to change facial expressions on asking patient to do movements exacerbating pain, e.g. range of motion, palpation, coughing, and so on. Voice data can be used to assess mood, affect, patent airway, hoarseness, and so on. Speech data can be evaluated for tone, paucity of words, flight of ideas, pressured, aphasia, work of breathing, interruptions for pain, lethargy, and so on. Eye tracking data can be used to estimate age, gender, and ethnicity and assess degree of fatigue, acute intoxication status and chronic substance use habits (alcohol, amphetamines, cannabis, tobacco), emotional state and valency, mental workload, moments of distraction, as well as particular words that may have caused confusion for the patient.

The system can issue a triage alert if patient reaches a state of self-harm or harm to others, such as in preferring more violent games, or having increased frustration/anger levels. This information can also be elicited by providing measured irritation stimuli patient to see ow emotionally reactive the patient becomes. The information can also elicited through word games or imagery games to gauge interest in self-harm or violence to oneself or others.

Smart health technology can support patients and family caregivers in managing complex, advanced symptoms. Smart health, comprising mobile and wireless technology, is increasingly recognized as an important tool to support personalized health care. Smart health has been shown to improve health outcomes for patients with a myriad of health conditions. A benefit of smart health is the ability to collect a wide range of relevant data passively, minimizing invasiveness and burden—an important consideration for patients and family caregivers coping with the stressors of advanced cancer. Leveraging smart health technology is extremely useful in understanding and characterizing the symptom experience in real time and optimally and holistically support patients and family caregivers. This can be combined with recording and tracking self-reported symptom data and communicating the results to healthcare providers.

When a patient or caregiver records a health event on their respective smartwatch, the application can provide a comprehensive snap shot of exactly what is occurring at and around the time of the event. This data can then inform and train personalized models that find relationships between behavioral, environmental, physiological, and contextual factors and pain events and implement real-time notifications for diagnosis, record keeping, or treatment. Recording symptoms in real-time enables accurate records, mitigating the effect time has on patient recall and accuracy.

The application can consider the role of multiple factors on the health event, including environmental, behavioral, physiological, and contextual factors. The application can explore how to best communicate shared data with key stakeholders how to best inform caregivers to manage the symptoms in the patient's setting, be it at home or a hospital.

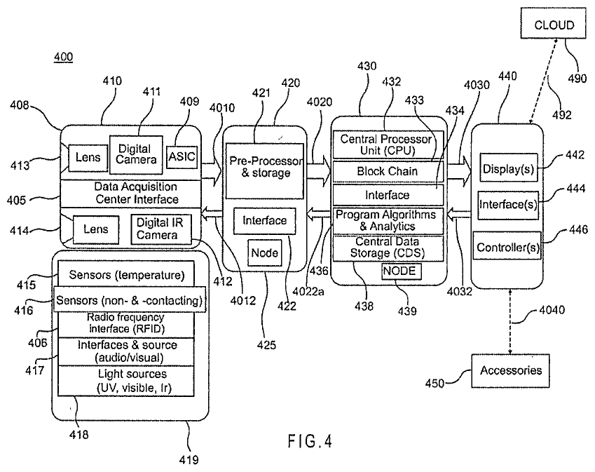

FIG. 4 represents a block diagram of a biometric imaging and biotelemetry (BIB) system 400, which is an intelligent and multifunctional biometric imaging apparatus configured to function as a medical diagnostic apparatus. The BIB system 400 includes a set of at least two, high-performance digital camera sub-systems 411 and 412 that may be enveloped within a suitable housing such as a Data Acquisition Apparatus (DAA) 410. The digital camera subsystems 411 and 412 are electrically connected to or have electrical communication with at least one Specialized Synthetic Intelligence (SSI) preprocessor 420, employing an advanced high-performance computing (HPC) and storage apparatus 421 and data interface 422. The SSI preprocessor 420 is electrically connected to or has electrical communication with at least one Specialized Synthetic Intelligence (SSI) central processor 430, employing an advanced high-performance computing (HPC) system 432. At least one application specific integrated circuit (ASIC) 409 is disposed within the DAA 410 and electrically connected to the digital camera sub-system 411. The BIB system 400 also includes at least one data node 425 and 439, where the data node 425 is operatively disposed in the SSI preprocessor 420, and where the data node 439 is operatively disposed in the SSI central processor 430. At least one custom Block-Chain protocol 433 is included with the SSI central processor 430. At least one digital display unit 442 is electrically connected to or has electrical communication with the SSI central processor.

Optionally, the DDA 410 is electrically connected to or is in electrical communication with a sensor subsystem 419, which includes at least one of a sensor 415, a detector 416, an audio source or receiver 417, and a light source 418 that is configured to function within at least in one part of the electromagnetic energy spectrum such as a narrow spectral region and an ultraviolet (UV) region. Optionally, at least one interconnecting, encoded linkage 492 interconnects the BIB system 400 with the “cloud” and related “cloud computing” 490. As a further option, at least one external system interface accessory 450 or electromechanical device is operatively connected to the at least one digital display 442. The BIB system may include one or more of a set of interconnecting wires, cables, fiber optics, radio frequency (Rf) receiver(s), RF transceivers, peripheral component interconnect (PCI), network cards, combinations, or the like which are not shown.

Diagnosis can integrate multiple sources of data. Some data can be provided directly by medical diagnosis equipment, such as measuring blood pressure. But data can additionally be obtained from more conventional sources, such as from wearable sensors, cell phone sensors, credit card data, search history, VR headsets, smart home sensors, computer and security cameras, and other electronic sources commonly used by the patient.

Smart watches can be worn by both the patient and the caregiver to collect both passive sensor data (such as heart rate and motion data) and active Ecological Momentary Assessment (EMA) data. Each smartwatch is programmed with a custom application designed for either the caregiver or the patient. The custom smartwatch app includes both event-triggered and scheduled EMAs. Event-triggered EMAs allow patients and caregivers to independently mark medical events and record pain severity, perceived distress, medication use, and use of non-pharmacological regimen.

Scheduled EMAs are automatically generated at designated intervals and ask a brief series of true/false or multiple-choice questions regarding mood, sleep quality, activity level, and amount of social interaction. Answers can be selected via the smart watch user interface or another interface available to the system.

Sensor relay stations are custom-built environmental sensor stations. Sensor relay stations are strategically deployed in each monitored room to passively and continuously collect data on room-level temperature, light, humidity, barometric pressure and ambient noise. Sensor placement is made in consideration of sensor range, communication range, and size/configuration of the room. In a preferred embodiment, environmental data streams are integrated and transmitted to the base station, for accumulation, interpretation, and transmission to the application data processor and/or database. However, note the base station can be integrated with the processing system and/or the database system, and the sensors can be connected by wired or wireless network, or connected directly into the system using a standard wired or wireless computer interface.

Bluetooth beacons are used to continuously broadcast device-identification information so that their broadcast signals are received by the smartwatches. Using the smartwatches' received signal strength indicator the application can determine the wearer's distance from each beacon, thereby enabling room-level localization of the wearer and an estimation of patient to caregiver proximity.

A Base Station can be a computer or another smart network device at the patient's location that provides a platform for data collection and transmission and a control for remote system monitoring.

Examples of useful data are quantity/quality of sleep, number of REM/NREM cycles, the heart rate, frequency, duration, and nature of exercise, hydration status, of exercise, diet quality (such as from credit card information on fast food purchases, impulsive purchases, etc), state of health (such as if the heart rate increases going up stairs, or presence of multiple heart rate accelerations per day indicating arrythmias, stressors, panic attacks).

The application can also collect contextual data, such as data pertaining to what is happening around the patient or caregiver or data measuring factors that can influence the patient or the caregiver experience. For example, this data can be a measure of appetite of the patient and/or caregiver, or a measurement of the energy level or fatigue level of the patient and/or caregiver, measure pain severity; level of patient activity, distress levels, sleep quality and quantity, mood, current location, time spent outside the patient residence, fatigue, appetite, social time spent with other people.

The system can use one or more sensors to monitor the patient or the environment. For example, thermo-sensors, olfactory sensors, vibration sensors, optical sensors, orientation sensors, motion sensors, posture sensors, motion sensors, and eye-tracking sensors.

These sensors can be directly controlled by the system, or be parts of diagnosis equipment which is connected to the system. Diagnosis equipment can be any equipment that finds use in medical diagnosis, such as digital endoscopes, oscilloscopes, blood pressure monitors, MRI, computer vision systems using visible light and IR cameras, head-mounted sensors and diagnosis gear, wearable sensors, blood condition monitors, and so on. For example, VR headset can be used to analyze an Alzheimer's risk by detecting amyloid plaque on retina, a proxy for amyloid plaques in the central nervous system).

Symptoms may also be extracted by processing a combination of data sources, by AI, by signal processing, or by a combination of the two. For example, a logical decision tree processing can be augmented by referencing AI processed data, such as by neural processing. Or, an AI can reference data provided by signal processing of selected data inputs, such as to extract useful information from general data. Of course, multiple AI and signal processing techniques can be combined in parallel and sequential combinations to perform diagnoses based on particular data.

The combination of data sources can be pre-processed by a local AI or algorithm to format the data for use with an external AI or signal processing resource. For example, reference data and collected data including sensory can be formatted into natural language queries for better application of a large language model AI, such as GPT.

Alternately, a large language model AI can be used to compress all the available reference data into a smaller set of key phrases or fingerprinted text elements that can be used to process the input information and data on a local processor or AI system.

Processing and sensory operations can be recursive. AI performs substantive data interpretation throughout the engagement with the patient's body and monitoring technologies, dedicating the application resources to assessing the data meaning and best methods of presentation for further computer, AI, or human evaluation.

Understanding the meaning of the biometric data is recursive. A physician senses the patient, the sensors sense the patient, the physician senses the sensors, the patient senses his own body, this data is collected and fed into a sensemaking algorithm and/or AI to determine the possible meanings of these indicators, and this process continues throughout the procedure.

The system can integrate a biometric imaging system that is configured to acquire a combined visible-light and thermal digital image of at least a portion of an individual, and decompose the combined digital image into individual and identifiable subregions where each subregion may be configured to represent at least one microscopic area of the individual. The image can be formed by an image capture process employing one or multiple selected regions of the electromagnetic spectrum.

The system can include one or more preprocessor subsystems configured to receive a digital data stream, such as data in the form of an image-configured data format, where the preprocessor serves to receive and convert the incoming data stream into a format suitable for mathematical digital processing. For example, the digital data stream can contain raw image data, compressed image data, enhanced image data, image data captured using different imaging conditions (for image enhancement and post-processing), and/or digitized biomarker data.

The collected data can be fitted into the data stream based on similarity to the fields allowed under the standard image format. For example two-dimensional data can be fitted to image data fields (such as fields for different colors, multi-views, interlace fields, and so on), and other data can be stored as flags or metadata.

The at least one central processing system is configured to receive at least one data stream in the format of one of a preprocessor transposed data and an image-formatted data, where the central processor has at least one software-based program with at least one mathematic algorithm that provides the central processor with a capability to at least one of analyze the data stream, select and extract data from the data stream for use in performance of at least one mathematical calculation or intelligent inference, draw intelligent conclusions from the calculation results, generate detailed and summary reports, and direct subsequent data and report outputs to at least one pathway interconnecting the central processor with encoding and decoding protocols for user interface devices and accessories, and for local or remote storage.

The central processing system can also be implemented as an AI system or a combination of AI, GPU, and CPU processing resources. Custom integrated circuits and other processing architectures can be similarly interfaced with the system for specialized or enhanced processing capability.

Large computations, such as computations based on different data sources or different portions of an image, can be partitioned and performed using distributed processing. Distributed processing systems of general purpose or special purpose computers, use a framework of common electrical, physical, and logical interfaces to specify and enforce compatible interfaces between software programs executing on those computers that perform discrete defined processing (components). The software system of components and framework can implemented on the network of distributed processors, where one or more software components execute on a processor. When the computation problem is such that using a distributed processing system of general purpose or special purpose computers requires more processing time and resources than can be provided in the processors, an alternative approach can be used. To obtain more processing per volume or per cost, the system can use specialized digital hardware such as FPGAs, gate arrays, or special purpose integrated circuits, connected using specialized interfaces.

The SSI preprocessor 420 consists of a Data Preprocessing Apparatus (DPA) 421 that serves to receive the biometric data from the DAA 410 via an appropriately configured interface 405 and interconnection 4010 and then via use of a specific DPA interface integrated together with a specific, intelligent and self-learning processor 421 along with appropriately configured algorithms (not shown) functions to calculate, compare, and contrast the incoming data against preprogrammed specifications, norms, and protocols that may be stored locally within the preprocessor 421. Within the DPA 421 is a provision to store selected data as prescribed by one, or more DPA algorithms. Then, at the same time, the DPA 421 serves to segregate portions of the incoming data from otherwise routine, secondary analytical processing when each data element has been determined to fall short of meeting preestablished specifications. The subset of segregated data may be appropriately formatted and passed directly on via at least one data interface 422 and interconnection 4020 and 4030 to a suitable display 442 or other data output accessory 450.

The Central Processing Unit (CPU) 430 receives the remaining data subset from the DPA 420 through a specially configured interface 434. Within the CPU 430, the incoming data is analyzed and processed by use of a Specialized Synthetic Intelligence (SSI) 432 processor. The CPU 430 is configured with advanced mathematical algorithms 436, executable instructions, at least one central data storage module 438 and an inter-module interface 434, at least one neural networking node 439 and various inter-subsystem interconnections 4020, 4022, 4030, and 4032.

The System Controller Unit (SCU) 440 receives the processed data as a subject-specific profile in the form of parameters, groupings of parameters, data matrices, one or more digitized data streams, mathematical functions, analytical conclusions, graphic relationships, operational instructions, combinations, and the like. The processed data may take on the form of any suitable formatting and may be encoded for optional external transmission 492 to the cloud 490 or the like. The SCU 440 is configured to perform the following functions: interface and interact with each of the subsystems and accessories; (2) enable programming and modifications to programs including downloading of externally generated programs to any designated subsystem; and (3) serve to direct and control the flow of data throughout the system. The BIB system 400 may also include multiple data pathways 4010, 4020, 4030, 4012, 4022, 4032, 4040, and 492 configured to communicate and share appropriately configured data and commands between and amongst the system subsystems.

The BIB system 400 may have one or more other scanners or sensors. The BIB system 400 may have at least one non-contacting iris scanner. The BIB system 400 may have at least one contact sensor 416 configured to acquire biometric data via direct, albeit non-invasive, contact with the test subject. This at least one sensor 416 enables the BIB system 400 to acquire, analyze, record, and integrate supplemental biometric data into a medical diagnosis as a means to help improve the accuracy of the resultant medical diagnosis. The at least one contact sensor 416 may comprise an identity badge reader, a credit card reader, a fingerprint reader, an oxygen sensor, a sphygmomanometer such as a blood pressure cuff, an ophthalmoscope, combinations, or the like.

The BIB system 400 may have at least one sensor (not shown) configured to acquire data, such as identification data related to an individual, via a device such as an employee identification badge, a credit card, a health or other insurance card, or a health-related file. The sensor may be configured to operate as a magnetic reader, a reflected light reader, a laser light reader, a bar chart reader, a glyph reader, combinations, or the like.

The BIB system 400 may have a sensor subsystem 419, which may include at least one interface 406 which may be a RFID configured interface, at least one sensor 415 and 416, and at least one source 418 integrated and contained within a suitable enclosure and located either integrated with, adjacent, adjoining, or remotely located from the camera module 408. The BIB system 400 may have at least one sound emitter or receiver 417, at least one light emitting source 418, combinations, or the like.

The BIB system 400 may have a Data Acquisition Apparatus (DAA) 410 that serves to sense and acquire biometric data from a test subject 110. The DAA 410 is comprised of at least one camera module 408 configured to house, encase, protect from damage, and enable positioning and mounting of at least one high resolution digital camera 411, at least one lens 413, at least one application specific integrated circuit (ASIC) device 409, and at least one data interface 405. The camera 411, the lens 413, the ASIC device 409, and the interface 405 may optionally be integrated into a self-contained assembly. The camera module 408 may also be configured to contain, encase, and protect from damage at least one thermal image sensor 412, related lens 414 and ASIC device 409.

The DAA 410 preferably uses imaging cameras and IR sensors having high pixel counts, ultra-high resolution (preferably 120 MP or more), and high operational and data transfer rates with faster readout speeds and with lower noise ratios preferably at 50 frames per second (fps) or higher. These and similar units are deemed to function suitably as components of the Data Acquisition Apparatus 410, which may have at least one camera member 411, at least one sensor member 412, and at least one aperture 413 and 414. Further sensing technologies are emerging that may also be suitable for deployment within the DAA 410. An example of a functional enhancement to a camera unit and ASIC component is a high precision temperature sensing member that is integrated within the camera unit that may serve to monitor intra-device temperatures and ambient temperatures, to reduce overall size of the camera module, and to increase data transmission efficiency.

The biometric sensory system can be implemented using two or more high-performance digital camera sub-systems, one or more Specialized Synthetic Intelligence (SSI) interface, an application specific integrated circuit (ASIC), a Block-Chain protocol, an interfacing operating system (OS) with a graphical user interface (GUI), an integration protocol for use on existing platforms, an optimization function for at least one of Data Structure, Big-0 notation, Deep-Learning, and Machine Vision, a set of interconnecting paths, where the interconnecting paths are at least one of wires, cables, fiber optics, and networking circuits, and a digital display.

The DAA 410 may be configured with a light source 418 having the capability to emit white or broad-spectrum light, or alternatively, to emit light having at least one preselected wavelength or range of wavelengths. The light may be unpolarized or polarized dependent upon the specific requirements of the diagnosis under assessment and/or an instruction set issued by the BIB system 400. In addition, the light source may be configured to emit various intensities of at least one designated wavelength or of a range of various and perhaps blended wavelengths to create a composite. The intensity of constituents of the composite may be constant or variable. Further, the light intensity may be constant over a designated period of time or variable as determined via designations of selectable light characteristics (e.g., wavelength(s), mix of wavelengths, intensities, and time intervals), which may be established by a suitably constructed data exchange between and amongst the DAA 410, the SSI preprocessor 420, and the CPU 430.

Emitted light from the light source 418 may be used to enhance and/or expand the diagnostic capabilities of the BIB system 400. The light source may be an appropriately configured multi-spectrum light emitting source having a blend of wavelengths and intensities that are modified to help reveal what otherwise may be invisible or nearly invisible biomarkers. For example, a multispectral light source having alternating high levels of the blue and red spectral regions may be used to illuminate the neck region of a test subject. Under these conditions, the carotid artery becomes quite visible when illuminated by a properly configured light source even if carotid artery is located below the surface layer of the neck. Subsequent imaging over a period of about a month reveals that a statistically significant enlargement in this artery has been observed and that the temporally-based enlargement may be correlated with the onset of hypertension in the subject patient. A similar finding has been observed when a major artery in the forearm region of a patient, who has been imaged and analyzed under similar conditions, and whereafter a clinical physician validated the pre-diagnosis and prescribed a plan of treatment.

In a further example, upon processing an initial stream of image data and conducting preliminary analyses on a test subject, an initial determination is reached by the AI processors that a particular group of ailments such as rosacea, COVID-19, flu, cold, or other suspected ailment may be distinguished by one or more facial colorimetric indicators, which may be present and employed as a biomarker(s). The at least one of the camera units 411 and 412 of the DAA 410 has served to acquire at least one facial image representing the entire colorimetric spectrum. The full-color spectrum data is delivered from the camera module(s) 411 and 412 to the Data Acquisition Center's interface 405, which serves to direct the image data stream to the SSI preprocessor 420. The SSI preprocessor validates the incoming data stream 4010 and then encodes the colorimetric image data into at least one computer processable data format, which may be referred to as machine augmented data. The SSI preprocessor 420 directs a selected portion of the data stream 4020 to the CPU 430 and working in concert perform a series of AI-enabled analyses that then establish a preliminary diagnosis. To arrive at the preliminary diagnosis, AI and algorithmic enabled comparatives are conducted by use of at least one of symptom-characteristics, medical records, case records, real-time updates, combinations, or the like that have been internally achieved as a library in the Central Data Storage (CDS) unit 438, and/or optionally provided via interconnection(s) with the internet and/or the cloud 490.

The processors' comparisons of color-specific biomarkers result in an issuance of a stream of commands that are conveyed to the light source unit 418, which serves to configure the spectral and temporal composition and emit a specifically configured light sequence to illuminate the test subject with at least one image coloring enhancement that may be employed in a follow-on image acquisition process and re-analysis of the color-enhanced image.

Such color enhancement may be particularly useful when the BIB system 400 has insufficient (or confusing) data to generate a clear diagnostic conclusion. An example may be the case where the initial facial imaging of a test subject records a somewhat elevated maximal temperature (Tmax) along with a number of reddish appearing regions. The regions are observed along both edges of the nose and within the eyes. The regions appearing as reddening of the white portion of both eyes believed to be symptomatic of blood vessels on the surface of the eyes that are expanded (i.e., dilated) due to a form of infection and not due to an artifact of the imaging process. A fresh set of images is acquired by the DAA 410 under various lighting conditions configured to emit certain light wavelengths for designated time periods.

A lighting sequence, for example may comprise at least one of a narrow blue spectrum component and at least one red spectrum component projected individually onto the subject for a similar brief period (e.g., 100 ms). The red-light component may serve to obscure, darken, or black-out the reddish regions while the blue-light component may serve to highlight the reddened regions and thus enable a better definition of the colored regions. The precise areas and color intensity of the reddened regions may thus be easier to precisely quantify and enter as such into the individual's profile for further reference. If upon subsequent evaluation after passage of time (e.g., 24 hours as in the case where an employee receives a routine daily evaluation prior to permitted entry into a workplace), changes to the reddened biomarkers may be observed and employed to aide in a medical diagnosis. Depending upon the nature, location, intensity of the changes, the system may display advisory messages to the individual, to the system administrator, and/or clinician, where upon appropriate action(s) may be taken to follow-up to the advisory message.

In an example where a slightly elevated maximal temperature (Tmax) and reddening of nose and eye regions have been observed, the light source may be employed to illuminate the test subject using a sequence of unpolarized light followed by polarized light. A comparison of the reflected images under these specific illumination conditions may better define a subregion immediately below the nose wherein a liquid-like substance may be observed and associated with a nasal discharge. The system may analyze this combination of quantified biomarker parameters consisting of Tmax, CRE, CLE, CNL, CNR, CDEL, CDER, CDNL, and CDNR along with the nasal discharge observation, and thus generate and display a diagnosis. Medical diagnoses delivered by a digital display member to a system user and thereby provide the user with high confidence insights into the wellness or types of illness that may be impacting upon a test subject. Given the enormity of data generated in this particular transaction, the BIB system may be configured with advanced algorithms that provide the capabilities to further mine the data and project that these particular symptoms better align with those of a particular ailment such as a cold as opposed to other ailments.

Finally, AI and/or signal processing can be used in implementing feedback loops to improve accuracy of the diagnoses or to calibrate the diagnosis system for the particular patient, the particular environment, the particular diagnosis equipment, or the particular sources of data used by the system.

Feedback can also be generated to train the AI based on additional diagnosis data. Training can adjust the method by which a particular diagnosis is performed, such as the manner in which sources of data are combined and processed to reach a diagnosis.

Alternately, AI can be used to calibrate the algorithms used in the data processing. This has the advantage of transferring the power of AI to a faster and less compute intensive processing.

Training can also adjust a way in which a particular source of data is used (such as if some sources of data prove more or less accurate for a particular patient), or to adjust weight given to a particular source of data in a particular diagnosis (such as when other sources become available or unavailable.

Finally, training can be used to reduce the number of sources of data used in each diagnosis based on the feedback data. Once the availability and the accuracy of the data sources can be evaluated, the training system can determine which data sources are required to make a diagnosis at a desired level of accuracy (at or above an accuracy threshold). Data sources that are deemed not required to the desired level of accuracy can be removed from processing or reduced to lower levels of processing or measurement. This is important in improving diagnosis time, in reducing the computing resources required for future diagnosis operations, in reducing power consumption, and in improving system stability in having fewer variables that can confuse the diagnosis algorithm or create a feedback instability.

Additionally, where expected data is unavailable (such as where the source of data is not available, is unreliable, or is malfunctioning), training can be used to add sources of data to the processing to compensate for the shortage of diagnosis data. For example, a different brand of smart watch can be poled for data, and training can be performed to adapt the diagnosis to use the new type and format of sensor data.

The system can further collect data using an optical diagnosis system that takes images of various light spectra, and processes the images to establish patient condition and perform or aid in a diagnosis. For example, such an optical system can be used to evaluate dry mucous membranes (dry mouth, tongue, etc.) It can also be used to evaluate patient's eyes for discharge, redness, bags under eyes, asymmetry, pupil size, reaction to light, direct and consensual eye response, etc.

It can evaluate patient's skin for conditions that can be visible on the surface of the skin, as well as subcutaneous conditions and deeper tissue and organ conditions. The images can be stimulated and recorded externally to the patient, or internally through use of endoscopes and pill-based sensors.

Various patient conditions may be made visible to the optical equipment by controlling illumination and image capture ad different wavelengths of light spectra, using different polarization effects, response to light sources of various wavelengths and or polarizations, or combination of such techniques that stimulate and capture optically detectable conditions.

Optical equipment can capture light that is reflected from the surfaces or sub-surface structures of a patient, but it can also capture effects of fluorescence, phosphorescence, luminescence, and transmission of light through the desired samples.

Patient can be diagnosed in vivo or directly or samples taken from a patient may be analyzed in vitro.

Below are examples of diagnoses that could be performed using the disclosed system:

Dentition—AI can alert based on physical appearance to evaluate for bulimia signs—dentition, spotting on teeth, irritated uvula, Ianugo hair—if risk high, AI can begin bulimia/anorexia evaluation asking patient if feels cold, dizzy on standing up (orthostatic hypotension), exercise amount, diet, etc.

Jugular venous distention, visible swelling in lower extremities, difficulty breathing—signs of heart failure. Evaluating hands & nails & toes, such as for arthritis deformities, pitting psoriasis, diagnose warts, fungus, etc. Ulcers, bruises, rashes. Evaluate musculoskeletal symptoms. Range of motion—how much hand can move, fingers, elbow, knees, toes, asymmetries in range of motion. Evaluate strength (e.g. symmetry in both arms when lifting household objects. Evaluate back pain, scoliosis. Asymmetry on bending forward. Degree patient can bend sideways, forwards, backwards, without pain. Does raising leg when lying down cause pain?

Any “Pain” question, the system can elicit information such as: was there a trigger at onset, anatomic location, acute, acute-on-chronic, or intermittent pain episodes, number and duration of episodes, characteristics of pain (sharp, dull, burning, etc), aggravating & ameliorating factors, radiation of pain to other locations, time course, severity, medications tried, has it happened before, difference in quality/severity from past occurrences.

“Location-based complaint”: elicit functional status of adjacent organ systems. For abdominal pain, for example, evaluate following organs: liver (yellowing of skin or eyes, color of stool), gallbladder (colicky pain associated with eating), esophagus (reflux), stomach (appetite, nausea, vomiting, weight loss), reproductive tract (pain on urination, pain with sexual, discharge, sexual practices, history of STIs), kidney (colicky pain, blood in urine), intestines (diarrhea, constipation, blood in stool), heme (pale conjunctiva).

Evaluate metabolic conditions: ask about urinary frequency, weight loss history, polydipsia, polyuria, nocturia, picture of urine if concentrated or dilute

For neurologic complaint (headache), evaluate: visual system (vision, nystagmus), vestibulo-cochlear system (hearing, balance, dizziness, vertigo), Cerebellar system (balance, dysdiadochokinesia), cranial nerves, motor strength, reflexes.

Define “triggers” and/or “modulating factors” (applicable for pain, shortness of breath, dizziness, etc.), exercise, changes in position, time of day, environment, stress, allergies, asthma, medications.

Identify risk factors or chronic states of health, such as heart conditions, lung conditions, autoimmune, endocrine, metabolic syndrome, smoking, personal or family history of cancer, diabetes, high blood pressure, high blood sugar.

Identify medication appropriateness for age, comorbidities, newly documented symptoms and conditions, and newly released medications, and updated information about medication use, interactions with patient's other medications, and patient's ability to metabolize and process the medication. The information can be entered into the system by a service provider, the patient, or the patient's caregiver. Updated medical and medication information can be downloaded from an appropriate database or publication, or can be sent directly to the patient's application by the healthcare service provider.

Identify signs of an infection: presence of runny nose, sore throat, ear ache, cough, with/without sputum and color (“secretion” subset questions), head ache, stomach ache, changes/pain/blood in stool or urine, etc. Also, patent's travel history and any sick contacts. Signs of secretion (e.g. phlegm, urine, stool, vaginal/penile discharge): color, consistency, odor, presence of blood, pus, mucous, associated itching, pain, redness, lesions in the area.

Skin evaluation. Rash, location, extent, appearance (color, borders, flat/raised, pustules, papules, vesicles, bullae, etc.), itching, redness, pain, warmth, swelling, fever, “infection” subset symptoms. Inquire about changes in detergent, jewelry, nickel or poison ivy contact. Bruises, trauma, wounds (clean, dry, intact, scabbing, scars). acne, eczema, nail pitting (psoriasis), and other dermatologic conditions.

Standard subset of H&P: past medical conditions, any events requiring overnight hospital stay, current medications, allergies and what happens if exposed to allergen, family history of conditions, surgical history (dates, complications), social habits (smoking/drinking/substance use/sexual practices, safe in relationships, mental state, stress levels), psychiatric history, etc.

Evaluate the face: facial asymmetry (Bell's palsy, transient ischemic attack, stroke), facial expressions (pain), rate of blinking, micro-expressions (truth-telling, anxiety, mood), acne, thin/thick/oily hair, male hair distribution on females (hormone status).

Evaluate secretions. Urine: concentrated, bloody, quantity mIs. Stool: color (gray, tarry, brown), size, shape (Bristol system), bloody, mucousy, floating, etc. Phlegm: green, yellow, blood-flecked, mucousy. Autonomic arousal: sweating, pupil dilation, goosebumps.

Systemic health: pallor, jaundice, scleral icterus. Cardiovascular: extremity perfusion status (presence of ulcers, cyanosis, varicose veins). Corkscrew hair (vitamin C deficiency).

The application can elicit data that is not directly provided.

Patients are not always able to verbalize their symptoms or even accurately sense what is bothering them. On the other hand, individual sensors or diagnosis apparata may only get a part of the whole picture. The benefit of using AI, is that all of this information can be processed together to create both a more accurate diagnosis of individual symptoms and a more accurate assessment of a bigger picture and larger cause of the symptoms.

In addition to standard diagnosis questions and tests, the patient can be asked related but indirect questions. The responses can be used to gage the patient's state of mind, pain threshold/sensitivity and so on. The responses to such questions can also be used to normalize the levels of patient's other responses to improve the diagnosis accuracy.

Questions can be customized for each patient, for the interaction with the application or to improve the human interaction between the service providers that use the application and/or the patients that use the application. Questions can be customized based on the determination of patient's state of mind, based on cultural norms, on the biological sex of the patient or the service provider using the application, on the previously recorded patient behavior, on quality and completeness of previous patient responses to that manner of questions, and other factors that influence the manner in which the patient responds to questions and tests.

Questions and interactions can also be built around placing the patient in a comfortable, cooperative, and/or a self-reflective state of mind. Questions that allow the patient more freedom to express his or her feelings and concerns in a broader sense, to bring up pleasant personal information (like anniversaries and kids), interactions that provide the patient with a pleasant feedback, and other manner of interactions that improve the patient's mood and disposition.

The patient can also be asked to perform related but indirect actions to elicit measurable responses. The actions can be simple or complex; the patient can swallow, follow commands, perform mini-mental status assessment (memory, naming ability), compensate for hidden challenges (subtle drag mouse on screen), and so on. The patient can be asked to perform facial movements: smile, lift eyebrows, protrude tongue, say “ah” (uvula deviation).

These actions can be combined with a neurologic exam; a vision exam such as object tracking, nystagmus, visual fields; a hearing exam such as from modulating audio input; a balance test, such as a Romberg test, a cerebellar function noise-to-point, or a rapid alternating action; as well as systemic information, capillary refill, skin turgor, pale conjunctiva, thin/oily hair, acne.

Images of the patient can be processed to isolate body language and facial expressions including micro-expressions when answering questions; this information can be used to verify the accuracy of patient's answers as well as to indicate additional information such as pain or discomfort in performing particular functions, evaluating motion ranges, face symmetry, body symmetry, reaction time, and so on.

The application can help fill out forms.

The application can provide information based on selection of the type of form being filled out. The type of form can be selected from a list of common forms or from a list provided by a word search.

A user can also input queries into the system to get recommended answers based on the available information about the patient.

The queries can be input through a standard text input interface such as a keyboard, a virtual keyboard, handwriting recognition, and so on. The queries can be input through voice recognition of user queries, downloaded electronically from the service provider, or otherwise obtained from the service provider using an electronic interface. The queries can also be recognized from a scan of the form or a digital picture of the form using OCR and text processing, recognized from a screen capture or a digital picture of the query on a screen using OCR and text processing, and so on.

Then downloaded or scanned forms are available to the application, it can automatically fill out recommended answers. The recommended answers can be marked based on the certainty of the answer. Marking can be performed through text formatting including use of highlighting and color. When the application does not have an answer to a queries, it can request the Applicant to input that information and recommend sources where such information could be found. Once the form is filled out in this manner it can be formatted for electronic delivery to the service provider or printed out.

Queries posed to the Applicant can be converted to audio, such as by a text-to-speech conversion or by selection of standard pre-recorded queries. User can request the application to rephrase or to explain the query for clarification.

User can select to translate the presented information, of the user interface, of the forms to be filled out, of the queries to the user. The translation can apply to the textual and to the audio information.

The application can translate the information on-the-fly, comprehend the user response in the user-preferred language (such as Spanish), and auto-fill the response in the service-provider language (such as English).

### General Applications

Although the above features are described as being provided to a patient, this application is not limited to a use by a particular person. The application can be used by a doctor or another service provider in performing a diagnosis or filling in patient's information into forms and electronic systems. The application can also be used to use the available patient information to assess prognosis, such as one-year mortality risk or risk of medical treatment, time-to-benefit from procedures, suggested changes to diet, exercise, and sleep schedule, physical therapy, and so on.

The service provider need not use the application as a primary source of the diagnosis. The application can be used in addition to standard medical tests and determinations to confirm or to indicate alternative symptoms to test and additional tests to be performed.

The application can also aid in diagnosing rare or otherwise hard to determine conditions, that can be then confirmed using standard tests. The application can be used as an early warning system for some conditions that are not tested during an ordinary doctor's examination, for example, insidious development of anemia, vitamin deficiencies, or the development of cognitive and emotional changes.

Doctors may find the application most useful in resolving uncertainties in other test results.

1. A system for assisting a patient in filling out a medical form, comprising:
a storage device;
one or more sensors;
a user interface;
a network module; and
a processor communicatively coupled to the storage device, the one or more sensors, the user interface, and the network module, wherein the processor executes instructions that are stored in the storage device and that cause the system to:
image the medical form using the camera;
process the image to recognize text of the medical form;
process the text of the medical form to identify requested information in the medical form;
if the requested information is historic information, access stored patient data in the storage device;
process all sources of patient data to identify answers to the requested information;
modify the text of the medical form to insert highlighted answers to the requested information in the corresponding parts of the text.

2. The system of claim 1 wherein the processor further causes the system to display the modified text of the medical form to the user.

3. The system of claim 1 wherein the processor further causes the system to send the modified text of the medical form to the service provider using the network module.

4. The system of claim 1, wherein one or more sensors comprise biometric sensors.

5. The system of claim 1, wherein one or more sensors comprise one or more smartphone sensors.

6. The system of claim 1 wherein if the requested information is about the patient's present state, collect patient data using the one or more sensors.

7. The system of claim 1 wherein if the requested information is about the patient's present state, access patient instructions from the storage device, wherein the patient instructions are related to the requested information.

8. The system of claim 1 wherein if the requested information is about the patient's present state,
access patient instructions from the storage device, wherein the patient instructions are related to the requested information;
provide the patient with the patient instructions one at a time using the user interface;
collect user-provided data as the patient executes the patient instructions.

9. The system of claim 8, wherein the user-provided data can be collected through the user interface.

10. The system of claim 8, wherein the user-provided data can be collected by using the one or more sensors.

11. The system of claim 8, wherein the user-provided data can be collected from a remote data source using the network module.

12. A processor executed method for assisting a patient in filling out a medical form, comprising:
imaging the medical form using the camera;
processing the image to recognize text of the medical form;
processing the text of the medical form to identify requested information in the medical form;
if the requested information is historic information, accessing stored patient data in the storage device;
processing all sources of patient data to identify answers to the requested information;
modifying the text of the medical form to insert highlighted answers to the requested information in the corresponding parts of the text.

13. The method of claim 12 further displaying the modified text of the medical form to the user.

14. The method of claim 12 further sending the modified text of the medical form to the service provider using the network module.

15. The method of claim 12 wherein if the requested information is about the patient's present state, collect patient data using the one or more sensors.

16. The method of claim 12, wherein if the requested information is about the patient's present state, access patient instructions from the storage device, wherein the patient instructions are related to the requested information.

17. The method of claim 12, wherein if the requested information is about the patient's present state,
access patient instructions from the storage device, wherein the patient instructions are related to the requested information;
provide the patient with the patient instructions one at a time using the user interface;
collect user-provided data as the patient executes the patient instructions.

18. The method of claim 17, wherein the user-provided data can be collected by using the one or more sensors.

19. The method of claim 17, wherein the user-provided data can be collected from a remote data source using the network module.

20. A non-transitory computer readable medium storing instructions that cause one or more processors to:
image the medical form using the camera;
process the image to recognize text of the medical form;
process the text of the medical form to identify requested information in the medical form;
if the requested information is historic information, access stored patient data in the storage device;
process all sources of patient data to identify answers to the requested information;
modify the text of the medical form to insert highlighted answers to the requested information in the corresponding parts of the text.
